(19) United States
(12) Patent Application Publication
(10) Pub. No.: US 2023/0154623 A1
(43) Pub. Date: May 18, 2023

(54) TECHNIQUES FOR PREDICTING DISEASES USING SIMULATIONS IMPROVED VIA MACHINE LEARNING

(71) Applicant: Fetch Insurance Services, Inc., Bolingbrook, IL (US)

(72) Inventors: Audrey RUPLE, Blacksburg, VA (US); Johannes Paul WOWRA, Darmstadt (DE); John K. GIANNUZZI, Wellesley, MA (US); Danna RABIN, New York, NY (US); Christian DEBES, Darmstadt (DE)

(73) Assignee: Fetch Insurance Services, Inc., Bolingbrook, IL (US)

(21) Appl. No.: 17/455,268

(22) Filed: Nov. 17, 2021

(51) Int. Cl. G16H 50/50; G06N 20/20; G16H 50/20

(52) U.S. Cl. CPC G16H 50/50; G06N 20/20; G16H 50/20

(57) ABSTRACT

A system and method for predictive disease identification via simulations improved using machine learning. A method includes applying at least one machine learning model to features extracted from data including animal characteristics data of an animal, wherein outputs of the at least one machine learning model include a plurality of disease predictor values, wherein each disease predictor value corresponds to a respective disease type of a plurality of disease types; running a plurality of disease contraction simulations based on the plurality of disease predictor values; generating disease contraction statistics based on results of the plurality of disease contraction simulations; and determining, based on the disease contraction statistics, at least one disease prediction for the animal.

### TECHNICAL FIELD

The present disclosure relates generally to disease prediction using machine learning, and more importantly to improving simulations used for disease prediction via machine learning.

### BACKGROUND

Predictive modeling in machine learning is the field of machine learning related to training models to output predictions. Machine learning is particularly well-suited to this task, since the lack of requirement to explicitly program the models allows for accounting for complex and varying factors. As more data becomes available, the potential for predictive models trained via machine learning becomes exponentially greater.

One particular area in which predictive modeling may be useful is for disease identification and, further, disease prediction used to provide personalized health solutions. Moreover, using machine learning to aid in learning about diseases in the realm of animals (e.g., pets such as dogs or cats) can allow for uncovering trends in animal diseases that have been yet unidentified. These uncovered trends may be very valuable for purposes such as, but not limited to, actuarial science, disease prevention, and disease mitigation.

In this regard, it is noted that more accurate disease prediction can be used to greatly improve health care for pets by providing access to information regarding potential diseases of individual pets, by altering pet care plans to avoid negative health outcomes and to overall improve pet health, and by observing broader trends in animal health outcomes.

Despite the great promise that predictive modeling via machine learning demonstrates in fields such as pet health, such modeling continues to face challenges in accurately uncovering causal relationships between combinations of animal attributes and diseases. Techniques for further improving accuracy of machine learning models used for disease prediction beyond obtaining better data or manually tuning weights of models are therefore desirable.

### SUMMARY

A summary of several example embodiments of the disclosure follows. This summary is provided for the convenience of the reader to provide a basic understanding of such embodiments and does not wholly define the breadth of the disclosure. This summary is not an extensive overview of all contemplated embodiments, and is intended to neither identify key or critical elements of all embodiments nor to delineate the scope of any or all aspects. Its sole purpose is to present some concepts of one or more embodiments in a simplified form as a prelude to the more detailed description that is presented later. For convenience, the term “some embodiments” or “certain embodiments” may be used herein to refer to a single embodiment or multiple embodiments of the disclosure.

Certain embodiments disclosed herein include a method for predictive disease identification via simulations improved using machine learning. The method comprises: applying at least one machine learning model to features extracted from data including animal characteristics data of an animal, wherein outputs of the at least one machine learning model include a plurality of disease predictor values, wherein each disease predictor value corresponds to a respective disease type of a plurality of disease types; running a plurality of disease contraction simulations based on the plurality of disease predictor values; generating disease contraction statistics based on results of the plurality of disease contraction simulations; and determining, based on the disease contraction statistics, at least one disease prediction for the animal.

Certain embodiments disclosed herein also include a non-transitory computer readable medium having stored thereon causing a processing circuitry to execute a process, the process comprising: applying at least one machine learning model to features extracted from data including animal characteristics data of an animal, wherein outputs of the at least one machine learning model include a plurality of disease predictor values, wherein each disease predictor value corresponds to a respective disease type of a plurality of disease types; running a plurality of disease contraction simulations based on the plurality of disease predictor values; generating disease contraction statistics based on results of the plurality of disease contraction simulations; and determining, based on the disease contraction statistics, at least one disease prediction for the animal.

Certain embodiments disclosed herein also include a system for predictive disease identification via simulations improved using machine learning. The system comprises: a processing circuitry; and a memory, the memory containing instructions that, when executed by the processing circuitry, configure the system to: apply at least one machine learning model to features extracted from data including animal characteristics data of an animal, wherein outputs of the at least one machine learning model include a plurality of disease predictor values, wherein each disease predictor value corresponds to a respective disease type of a plurality of disease types; run a plurality of disease contraction simulations based on the plurality of disease predictor values; generate disease contraction statistics based on results of the plurality of disease contraction simulations; and determine, based on the disease contraction statistics, at least one disease prediction for the animal.

### BRIEF DESCRIPTION OF THE DRAWINGS

The subject matter disclosed herein is particularly pointed out and distinctly claimed in the claims at the conclusion of the specification. The foregoing and other objects, features, and advantages of the disclosed embodiments will be apparent from the following detailed description taken in conjunction with the accompanying drawings.

FIG. 1 is a network diagram utilized to describe various disclosed embodiments.

FIG. 2 is a flow diagram illustrating a multi-stage machine learning approach to predictive disease identification according to an embodiment.

FIG. 3 is a flowchart illustrating a multi-stage machine learning method for predictive disease identification according to an embodiment.

FIG. 4 is a flowchart illustrating a method for determining a predictions for different temporal ranges according to an embodiment.

FIG. 5 is a schematic diagram of a disease predictor according to an embodiment.

### DETAILED DESCRIPTION

It is important to note that the embodiments disclosed herein are only examples of the many advantageous uses of the innovative teachings herein. In general, statements made in the specification of the present application do not necessarily limit any of the various claimed embodiments. Moreover, some statements may apply to some inventive features but not to others. In general, unless otherwise indicated, singular elements may be in plural and vice versa with no loss of generality. In the drawings, like numerals refer to like parts through several views.

In light of the challenges and desired improvements noted above, techniques for improved predictive disease modeling as described herein have been developed. In particular, it has been identified that factors which influence diseases contracted by animals can be reflected in both broader categories of factors which have large sample sizes (e.g., sex, breed, common diseases, etc.) as well as narrower categories of factors with smaller sample sizes (e.g., specific ages, defined geographic locations, rare disease outcomes, etc.). Consequently, it has been identified that performance of predictive disease modeling for animals using machine learning can be improved by utilizing both models which perform better for larger sample sizes and models which perform better for smaller sample sizes. To this end, the disclosed embodiments include a multi-stage machine learning process that uses a combiner model to combine outputs from different individual models and, in particular, individual models that have different properties and therefore perform differently for different sample sizes, in order to provide more accurate estimations of probabilities for contracting diseases and, consequently, improved disease predictions.

It has further been identified that there can be a need to generate predictions relative to different time periods in order to anticipate future health conditions of animals. For example, the ability to predict likelihood of contracting a given disease within 1 year, 2 years, 3 years, and the like, may allow for adjusting actuarial estimates in the insurance context. As another example, such ability may allow for determining urgency of certain diseases, which in turn can be utilized to prioritize treatment steps and to determine how dramatically treatment should be adjusted. As a non-limiting example, for a dog that is likely to contract diabetes within 1 year, losing weight may be a prioritized treatment step such that it is recommended to begin immediately, and the amount of weight to be lost within 6 months may be higher than the amount of weight to be lost within 6 months for a dog that is likely to contract diabetes within 5 years. As yet another example, prediction of diseases at different stages in an animal's life (i.e., in different time periods) may allow for identifying potentially fraudulent insurance claims by comparing predicted diseases for an animal to diseases indicated in insurance claims for the animal.

It has yet further been identified that the number of known potential diseases for animals include over 1000 distinct diseases, as well as variations which may be too numerous to individually identify. Consequently, it has been determined that predefining groups of diseases in order to group similar diseases allows for improving machine learning techniques such as techniques for classifying diseases. More specifically, limiting the number of potential outcomes to predefined groups of similar diseases instead of each distinct disease allows for striking a balance between machine learning richness with accuracy of results. Additionally, reducing the number of classes predicted reduces complexity of the model, which in turn reduces computational resources needed to apply the model.

Similarly, the numbers of potential breeds for different kinds of animals can be enormous, with new breeds being created and bred over time. Thus, it has also been identified that predefining groups of breeds and grouping similar breeds of animals based on those predetermined groups allows for improving machine learning processes that utilize breeds as inputs. More specifically, by grouping breeds with similar genetic ancestry and using the predefined groups as inputs to a machine learning process, the machine learning process will yield overall more accurate results, particularly when breeds used as inputs include rare breeds or otherwise specific breeds which were not well-represented individually in training data.

To this end, the various disclosed embodiments include techniques for predictive disease identification using machine learning. In an embodiment, one or more machine learning models trained to output at least disease predictor values for classifications representing different types of diseases based on at least animal characteristic features are applied to a set of animal characteristic features of an animal for which diseases are to be predicted.

Based on the output of the machine learning models, predictions indicating at least one or more diseases that the animal is likely to contract in the future are determined. The outputs of the machine learning are used to run multiple disease contraction simulations for each temporal variation of a set of multiple temporal variations. Based on those simulations, disease contraction statistics are generated. The disease contraction statistics are utilized to generate predictions about the likelihood of the animal contracting each predicted disease within different periods of time, thereby allowing for determining predictions that further indicate diseases the animal is likely to contract in different periods of time. The predictions may further be utilized to generate recommendations, insights, or both.

In some embodiments, the machine learning models are applied in stages, with at least a first stage including applying an ensemble of machine learning models. The output of each model of the ensemble is input to a combiner model, which is trained to output disease predictor values for one or more diseases based on the outputs of the ensemble models. Based on the output of the combiner model, predictions indicating at least one or more diseases that the animal is likely to contract in the future are determined. In a further embodiment, determining these predictions includes running simulations based on the output of the combiner model. In another embodiment, the predictions may be determined based on the output of the combiner model without running simulations.

By utilizing the outputs of the machine learning models to run simulations of disease contraction scenarios which are also to be used for generating predictions, such simulations are run based on more accurate input parameters, thereby improving performance of the simulations themselves. This allows for generating more accurate statistics which, in turn, can be used to further improve accuracy of predictions. Moreover, applying such simulations on top of machine learning modeling allows for improving granularity of predictions as discussed above, namely, by accounting for temporal variations that allow predictions to be accurately estimated for different periods of time. Additionally, since the simulations in at least some embodiments are run based on a limited set of disease categories (i.e., a predetermined set including predefined groups of diseases), the complexity of the simulations can be reduced, which allows for running simulations more efficiently as compared to running simulations based on all potential types of individual diseases.

Further, the combined machine learning process described in accordance with various disclosed embodiments allows for increasing accuracy of disease predictions as compared to simply utilizing individual models to generate predictions, and also allow for improving accuracy of disease predictions as compared to an explicitly programmed combiner algorithm.

The result of the above is that the processes described herein demonstrate more accurate and more granular predictions than predictions made manually by veterinarians. Further, the disclosed embodiments provide an objective process for combining results of learned modeling and for predicting likelihoods of contracting diseases within different time periods which do not rely on the subjective judgments and anecdotal experience that come with manual disease prediction by such medical professionals. Consequently, the disclosed embodiments also provide more consistent results as compared to such manual techniques.

In addition to the various technical improvements noted herein, the improved accuracy predictions described herein can be utilized to improve pet care. As a particular example, more accurately predicting disease allows for increasing accuracy of financial analyses of risk such as work typically done by actuaries for insurance purposes. Moreover, the improved granularity afforded due to the temporal variations of predictions described herein allows for more accurately forecasting insurance rates over time. Thus, the disclosed embodiments can be applied in the pet insurance context in order to set pricing accordingly and to improve coverage offered to pets.

Additionally, by more accurately identifying diseases that pets are likely to have, suggestions for actions to avoid such diseases can be made more accurately. Further, the temporal variations of predictions allow for better determining relative urgencies of diseases, particularly when considering both temporal likelihoods of disease contraction and disease severity. Consequently, the disclosed embodiments may also be utilized in the clinical context in order to determine courses of action to prevent or mitigate disease, thereby improving animal health outcomes.

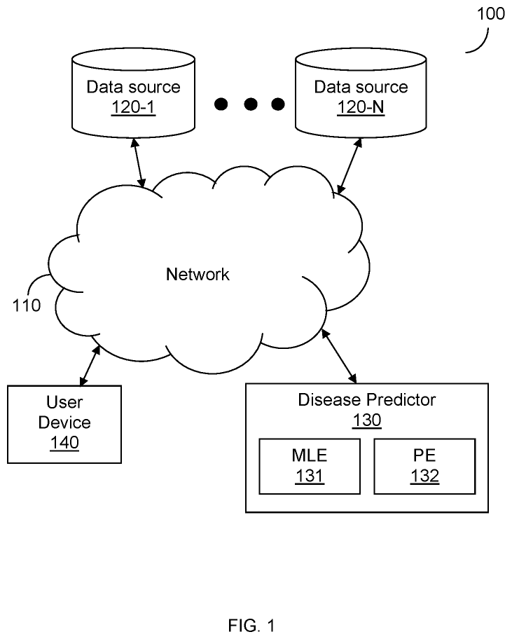

FIG. 1 shows an example network diagram 100 utilized to describe the various disclosed embodiments. In the example network diagram 100, a plurality of data sources 120-1 through 120-N (hereinafter referred to individually as a database 120 and collectively as data sources 120, merely for simplicity purposes), a disease predictor 130, and a user device 140 communicate via a network 110. The network 110 may be, but is not limited to, a wireless, cellular or wired network, a local area network (LAN), a wide area network (WAN), a metro area network (MAN), the Internet, the worldwide web (WWW), similar networks, and any combination thereof.

The data sources 120 store data to be used for generating disease predictions and may include, but are not limited to, one or more databases (e.g., databases storing clinical data for animals), data sources available via the Internet or other networked systems, both, and the like. The data stored by the data sources 120 may include, but is not limited to, disease data, animal characteristic data, environmental data, other external factor data, combinations thereof, and the like. Such data may be in the form of textual data, visual data (e.g., images or videos), and the like.

The disease data includes data related to diseases contracted by animals, and may further include time data indicating times at which the animals contracted certain diseases (e.g., as defined with respect to animal age). In some implementations, diseases indicated in the disease data may be grouped into predefined groups of similar diseases such that, when features are to be extracted from the disease data, specific diseases indicated by the disease data are first identified and then an applicable group of the predefined groups may be selected for each specific disease.

The animal characteristic data includes data for individual animals which may be related to disease contraction such as, but not limited to, breed, sex, age, geographic location, breed characteristics (e.g., appearance, grooming, exercise, nutrition needs, temperament, etc.), disease history, claim history (e.g., insurance claims, which may be grouped by disease type), claim costs, neutering status, pregnancy status, weight, potential symptoms of diseases (e.g., lesions, vomiting, etc.), activity tracking data (i.e., data indicating activities engaged in by animals), combinations thereof, and the like.

Breeds of the animal characteristic data may be grouped into predefined groups of similar breeds such that, when features are to be extracted from the animal characteristic data, specific breeds indicated by the animal characteristic data are first identified and then an applicable group breeds may be selected from the predefined groups of breeds for each of the identified specific breeds.

The environmental data includes data for environments in which animals live which may be related to disease contraction and may include, but is not limited to, climates of different geographic locations, relevant geographic structures (e.g., bodies of water), wildlife statistics (e.g., statistics indicating presence of other animals in the animal's environment), characteristics of a home in which an animal lives (e.g., house, apartment, etc.), combinations thereof, and the like.

The disease predictor 130 is configured to generate disease predictions as described herein. Such predictions are generated based on outputs of a multi-stage machine learning process that combines outputs from different models into disease predictor values for different types of diseases (e.g., specific types of diseases or groups of related diseases). To this end, the disease predictor 130 may include a machine learning engine (MLE) 131. The MLE 131 is configured to apply machine learning models in the multi-stage machine learning process as described herein, and may further be configured to train such models. Alternatively, another system (not shown) may be configured to train the models such that the models are trained as described herein.

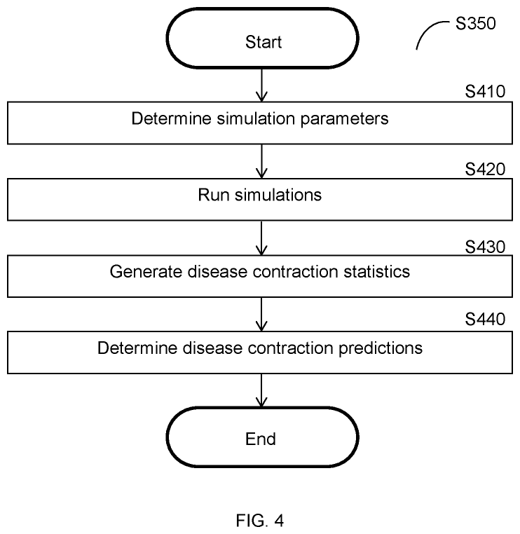

The disease predictor 130 is further configured to determine predictions based on the outputs of the multi-stage machine learning process. To this end, the disease predictor 130 includes a prediction engine (PE) 132 configured to generate predictions as described herein. The predictions may further be based on simulations also described herein and, accordingly, the prediction engine 132 may be further configured to run such simulations (for example, as described below with respect to FIG. 4). In some implementations, the disease predictor 130 may further include a recommendation engine (not shown) configured to generate recommendations for actionable tasks to perform with respect to disease predictions for animals.

The user device (UD) 140 may be, but is not limited to, a personal computer, a laptop, a tablet computer, a smartphone, a wearable computing device, or any other device capable of receiving and displaying notifications. In an example implementation, the user device 140 is of a user who owns an animal as a pet. The user of the user device 140 may provide characteristics of their pet, the environment in which the pet lives, and the like, as user inputs to be used by the disease predictor 130 to predict diseases. The user device 140 may send these user inputs to the disease predictor 130, and may receive notifications to be displayed indicating disease predictions, recommendations, insights, or combinations thereof, from the disease predictor 130.

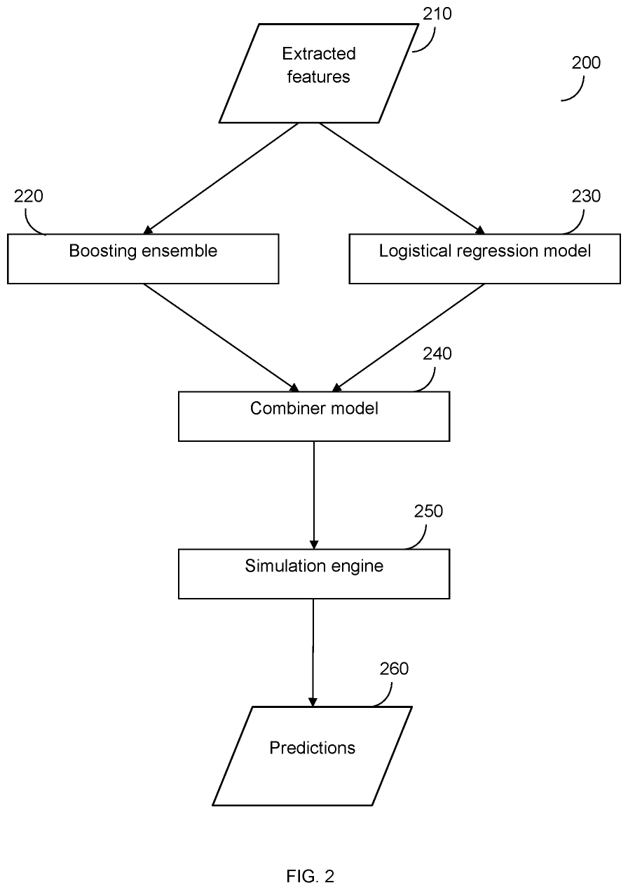

FIG. 2 is a flow diagram 200 illustrating a multi-stage machine learning approach to predictive disease identification according to an embodiment.

In an embodiment, features 210 extracted from data related to an animal are input to a first stage of machine learning models. In the embodiment depicted in FIG. 2, the first stage of machine learning models includes a boosting ensemble 220 and a logistic regression model 230 such that the features 210 are input to both the boosting ensemble 220 and to the logistic regression model 230.

The boosting ensemble 220 is an ensemble of sequentially applied boosting machine learning models (models of such a boosting ensemble being referred to herein as boosting machine learning models, not depicted in FIG. 2) trained using a boosting algorithm. Such a boosting algorithm sequentially trains models of the ensemble, where misclassifications by a model in the sequence made during training are used to adjust weights of subsequent models in the sequence. A boosting algorithm operates based on the principle of combining predictions of multiple weak learner models in order to form one strong rule for making predictions. In an embodiment, the output of the boosting ensemble is a disease predictor value (e.g., a probability) for each potential outcome, where each potential outcome is a disease type (e.g., a particular disease or a predefined group of diseases). It is noted that boosting ensembles tend to make predictions more accurately when applied to data from large sample sizes.

The logistic regression model 230 is a machine learning model trained to output a dependent variable with a finite number of potential outcomes. As a non-limiting example, a binary regression model outputs either A or B. As another non-limiting example, a multinomial regression model outputs one of a set such as A, B, C, or D. In an embodiment, the output of the logistic regression model is a disease predictor value (e.g., a probability) for each potential outcome, where each potential outcome is a disease type (e.g., a particular disease or a predefined group of diseases). It is noted that logistic regression models tend to make predictions more accurately when applied to small sample sizes.

In an embodiment, each of the boosting ensemble 220 and the logistic regression model 230 is trained to output a disease predictor value for each potential outcome (e.g., each type of disease which may be contracted by an animal), where the potential outcomes for both the boosting ensemble and the logistic regression model are the same set of potential outcomes. As a non-limiting example, when the potential outcomes include 70 distinct predefined groups of diseases representing 70 different disease types, each of the boosting ensemble and the logistic regression model may be trained to output a probability for each of the 70 predefined groups of diseases.

It should be noted that, at least in some embodiments, other types of machine learning models may be utilized during the first stage of machine leaning model application, either in addition to or instead of either the boosting ensemble 220 or the logistic regression model 230. In particular, other models which tend to demonstrate high accuracy for larger sample sizes may be utilized in addition to or instead of the boosting ensemble 220, and other models which tend to demonstrate high accuracy for smaller sample sizes may be utilized in addition to or instead of the logistic regression model 230.

The combiner model 240 is trained to utilize outputs of the first stage machine learning models 220 and 230 and in order to output a disease predictor value for each potential outcome, where each potential outcome is a disease type (e.g., a particular disease or a predefined group of diseases).

Given the above properties of boosting ensembles and logistic regression models, in an embodiment, the combiner model is trained to utilize outputs from a boosting ensemble with outputs from a logistic regression model in order to output a single set of disease predictor values. The result of this combination is a combiner model which accounts for variations due to both large and small sample sizes in order to more accurately predict diseases. In this regard, it has been identified that the combination of a boosting ensemble and a logistic regression model yields particularly accurate results in the context of disease prediction for pets and other non-human animals.

The outputs of the combiner model 240 are provided to a simulation engine 250 configured to determine predictions 260 of disease for animals. In a further embodiment, the simulation engine 250 may be further configured to output risk scores for a given animal contracting certain types of diseases (e.g., risk scores determined based on the probability of contracting each disease type), and to include those risk scores with the predictions 260.

In various embodiments, the simulation engine 250 may be further configured to perform simulations in order to determine temporal variations of disease prediction as described further herein, for example, as described with respect to FIG. 4.

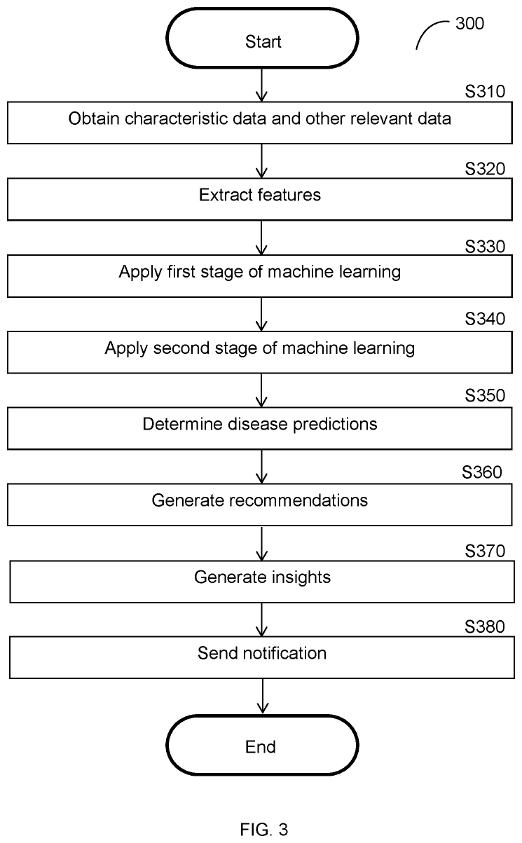

FIG. 3 is a flowchart 300 illustrating a multi-stage machine learning method for predictive disease identification according to an embodiment. In an embodiment, the method is performed by the disease predictor 130, FIG. 1.

At S310, animal characteristic data and other data to be used for determining disease predictions for an animal are obtained. The data may be received (e.g., from a user device such as the user device 140, FIG. 1) or may be retrieved (e.g., from a data source such as one of the data sources 120, FIG. 1). When the data is retrieved, such retrieval may be based on an identifier of the animal for which predictions are to be determined.

At S320, features to be used as inputs to the first stage of machine learning are extracted from the data obtained at S310.

In an embodiment, S320 may further include enriching the data obtained at S310 in order to provide more features to be used for the first stage of machine learning. Enriching the data may include, but is not limited to, retrieving relevant data based on other obtained data, inferring new data based on the obtained data, both, and the like. As non-limiting examples, climate data may be retrieved based on geographic locations indicated in the obtained data (i.e., climate data for those geographic locations is retrieved), neutering status or other medical records may be retrieved based on an identifier of an animal, claim history and costs may be retrieved based on an identifier of an animal, and the like.

In embodiments where enriched data is at least partially inferred, such inferences may be derived using machine learning. To this end, S320 may include applying a machine learning model trained to infer enrichment data using historical data and historical enrichment data. As a non-limiting example, such a model may be trained to output a classification of sex (e.g., male or female) based on inputs including (but not necessarily limited to) animal name.

At S330, a first stage of machine learning is conducted using the extracted features. The first stage of machine learning includes applying multiple machine learning models of different types. Each model or combination of models (e.g., an ensemble including a subset of models) among the multiple machine learning models ultimately outputs a respective first disease predictor value for each potential disease type (e.g., potential classifications of the models) to be input to a combiner model as described below with respect to S340.

In an embodiment, the first stage of machine learning includes applying a boosting ensemble, a logistic regression model, or both, to the extracted features or a portion thereof. The types of models applied during the first stage of machine learning are different such that, for example, when a boosting ensemble is applied during the first stage of machine learning, at least one non-boosting model is also applied during the first stage of machine learning and, when a logistic regression model is applied, at least one non-logistic regression model is also applied. As noted above, boosting ensembles and logistic regression models perform differently with different sample sizes of data such that using both types of models allows for more accurate outputs when applied to datasets of varying sample sizes such as datasets related to animal characteristics (i.e., since some animal characteristics are more common than others and therefore are demonstrated in larger sample sizes).

In a further embodiment, any or all of the machine learning models applied during the first stage of machine learning are supervised learning models trained to output disease predictor values for certain disease types in which the training of those supervised learning models uses a labeled training set. Such a labeled training set includes training input data (e.g., data indicating animal characteristics, environmental factors, etc.) as well as predefined training labels representing the “correct” outputs for respective combinations of training input data.

At S340, a second stage of machine learning is conducted using the outputs of the first stage of machine learning models. In an embodiment, the second stage of machine learning includes applying a combiner model to the outputs from the machine learning models of the first stage of machine learning. The combiner model is trained to combine outputs from the first stage of machine learning models in order to output a second disease predictor value for each potential disease type. To this end, the combiner model includes respective weights for the different models or ensembles utilized in the first stage of machine learning. Like the models applied during the first stage of machine learning, the combiner model may be trained via a supervised machine learning process using labeled training data including output training labels indicating disease predictions associated with different combinations of training inputs.

At S350, one or more disease predictions are determined for the animal based on the output of the second stage of machine learning. In an embodiment, each disease prediction may indicate a disease type (e.g., a specific disease or a predefined group of diseases) that the animal is likely to contract.

Alternatively or collectively, the disease predictions may indicate the likelihood of contracting certain diseases (e.g., as defined with respect to the disease predictor values output by the combiner model). In a further embodiment, an animal is likely to contract a disease when the disease predictor value for that disease output by the combiner model during the second stage of machine learning is above a predetermined threshold. As a non-limiting example where the disease predictor value is a probability, an animal may be determined to be likely to contract a disease when the probability of contracting the disease is above 60% (i.e., 0.6). To this end, in some embodiments, S350 may further include generating risk scores for each disease type based on the disease predictor values output by the combiner model.

Each risk score may indicate, for example, a degree of risk of the animal contracting the disease type (e.g., a risk score in the range of 1 to 10, with 1 being low risk and 10 being high risk). The risk scores may include risk scores indicating likelihood of the animal contracting a disease within its lifetime (e.g., based on an average lifespan of animals having the same or similar characteristics), risk scores indicating likelihood of the animal contracting a disease within a certain time period (e.g., within 3 years from now), both, and the like.

In another embodiment, determining the disease predictions may further include running simulations for the animal based on the disease predictor values output at S340. In a further embodiment, the simulations may be performed with respect to different periods of time such that the results of the simulations may be utilized to determine disease predictions for the same animal with respect to those different time periods. This, in turn, allows for providing increased granularity disease predictions.

An example method for determining disease contraction predictions and, in particular, disease contraction predictions with respect to different time periods, using simulations is now described with respect to FIG. 4. FIG. 4 is a flowchart S350 illustrating a method for determining predictions for different temporal ranges according to an embodiment.

At S410, simulation parameters are determined. The simulation parameters define how the simulations are run, and may be determined at least partially based on probabilities or other disease predictor values indicating the likelihood of an animal contracting certain diseases in combination with predetermined rules for determining simulation parameters using those disease predictor values. The simulation parameters include time periods for which simulations are to be run (e.g., within 1 year from present, within 2½ years from present, between 2 years and 3 years from present, etc.).

In an example implementation, the simulations may be Monte Carlo simulations. To this end, in some embodiments, S420 may further include assigning multiple values to variables used for the simulations based on disease predictor values for contracting different diseases (e.g., probabilities output by the combiner model as described above with respect to S340).

Monte Carlo simulations predict a set of outcomes based on an estimated range of values versus a set of fixed input values. For any variables with uncertain values, a model of possible results is created by utilizing a probability distribution to identify such potential results. Then, a Monte Carlo experiment can be run by running many simulations to produce a large number of likely outcomes. To this end, in an embodiment, S420 may further include determining a probability distribution for each potential disease type based on a disease predictor value corresponding to the disease type (e.g., probabilities output by the combiner model as described above with respect to S340) and creating a model of possible results for each disease type using the respective probability distribution for that disease type.

At S420, disease contraction simulations are run using the determined simulation parameters. In an embodiment, S420 includes running at least a predetermined number of simulations (e.g., 1,000 simulations) such that a large number of likely outcomes may be determined.

In this regard, it is noted that Monte Carlo simulations can be effectively leveraged for long-term predictions since such simulations exhibit increased accuracy for outcomes (even outcomes with projections that are farther out in time) as the number of inputs increase. Thus, Monte Carlo simulations provide the ability to accurately predict outcomes over time such that it has been identified that Monte Carlo simulations can be utilized to provide accurate temporal forecasting in accordance with the disclosed embodiments.

At S430, disease contraction statistics are generated based on the outcomes of the disease contraction simulations. The disease contraction statistics may include, but are not limited to, mean, standard deviation, both, and the like. Moreover, the disease contraction statistics are defined with respect to different time periods such that the statistics can be utilized to predict likelihood of contracting diseases in the different time periods.

At S440, predictions of disease contraction are generated for the animal based on the disease contraction statistics. As a non-limiting example, the likelihood that the animal contracts a given disease during a given time period may be determined at least based on the average

Returning to FIG. 3, at optional S360, one or more recommendations are generated based on the determined disease predictions. Each recommendation is an individualized recommendation for improving pet health and/or avoiding undesirable health outcomes such as contracting certain diseases or mitigating the severity of diseases the animal is likely to contract. To this end, the recommendations may include actions to be taken with respect to the animal such as, but not limited to, losing weight, changes in diet, and the like.

At optional S370, one or more insights may be generated based on disease predictions for multiple animals. In an embodiment, S370 includes comparing between the disease predictions for multiple animals to actual results (i.e., historical diseases actually contracted by those animals). To this end, in such embodiments, steps S310 through S350 may be repeated for multiple iterations (each iteration providing predictions for a respective animal based on input data related to that animal), and the analysis at S370 is based on the aggregated results of those iterations. Moreover, the iterations may utilize animals with similar characteristics (e.g., same species, same sex, same or related breed, same weight, similar environment, combinations thereof, etc.) such that trends can be based on like comparisons.

By comparing between predicted results and actual results, trends representing changes in disease contraction can be identified, which in turn allows for generating insights that demonstrate broader trends reflected in aggregated differences between what would normally be expected and what actually occurred. To this end, in some embodiments, S370 includes comparing results of simulations (e.g., the simulations run as described with respect to FIG. 4) run with respect to certain time periods to actual results for those time periods.

By comparing predicted results to actual results for a time period in which certain events occur, trends which may correlate with or be caused by that event can be unearthed. As a non-limiting example, by comparing predicted results for the time period between March 2020 and March 2021 which represents the first year of the novel Coronavirus pandemic to actual results for that same time period, trends in animal health which may be related to the pandemic may be identified. Such trends may include, for example, increases in insurance claims compared to expected claims during the time period in question, decreases in certain behavioral diseases during the time period in question, combinations thereof, and the like.

At optional S380, a notification may be sent. The notification may indicate, but is not limited to, the disease predictions, the recommendations, the insights, a combination thereof, and the like. The notification may be sent to a user device (e.g., the user device 140, FIG. 1), for example, a user device of a user who owns a particular animal as a pet or a user device of an administrator or other person who wishes to receive insights related to broader trends among animals.

It should be noted that the steps of FIG. 3 are depicted in a specific order for example purposes, but that the steps of FIG. 3 are not necessarily limited to the order depicted therein. In particular, steps S360 and S370 may be performed in any order or in parallel without departing from the scope of the disclosure.

Additionally, it should also be noted that FIG. 3 depicts a single iteration of disease prediction merely for simplicity purposes, and that multiple iterations of disease predictions may be performed without departing from the disclosed embodiments. These iterations may be performed sequentially (e.g., multiple disease predictions for the same animal or for different animals), in parallel (e.g., disease predictions for multiple different animals), both, and the like.

Sequentially performing iterations allows for, among other things, updating disease predictions, for example as new data about the animal becomes available. As a non-limiting example, whenever a disease prediction is required (for example, when a new insurance claim is submitted), a new disease prediction may be made based on the current data for the animal to ensure that the new disease prediction is based on up-to-date data. As another non-limiting example, new disease predictions may be determined through subsequent iterations when new data about the animal becomes available or otherwise when the animal characteristics or other data related to the animal is updated. Such changes may include, but are not limited to, updates to the animal's location (e.g., when the animal's owner moves), when a previously unknown sex of the animal has been determined, when the animal has been spayed or neutered, when a breed of the animal is updated, combinations thereof, and the like.

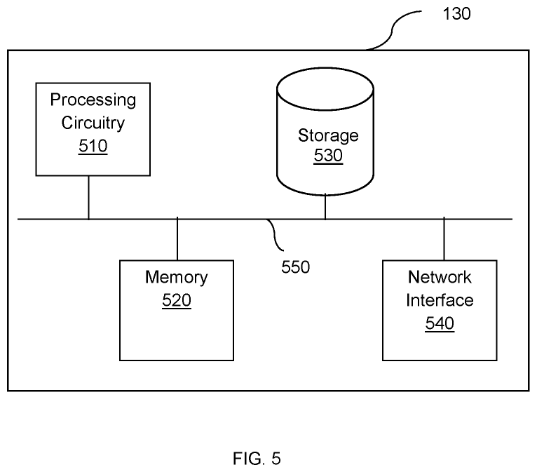

FIG. 5 is an example schematic diagram of a disease predictor 130 according to an embodiment. The disease predictor 130 includes a processing circuitry 510 coupled to a memory 520, a storage 530, and a network interface 540. In an embodiment, the components of the disease predictor 130 may be communicatively connected via a bus 550.

The processing circuitry 510 may be realized as one or more hardware logic components and circuits. For example, and without limitation, illustrative types of hardware logic components that can be used include field programmable gate arrays (FPGAs), application-specific integrated circuits (ASICs), Application-specific standard products (ASSPs), system-on-a-chip systems (SOCs), graphics processing units (GPUs), tensor processing units (TPUs), general-purpose microprocessors, microcontrollers, digital signal processors (DSPs), and the like, or any other hardware logic components that can perform calculations or other manipulations of information.

The memory 520 may be volatile (e.g., random access memory, etc.), non-volatile (e.g., read only memory, flash memory, etc.), or a combination thereof.

In one configuration, software for implementing one or more embodiments disclosed herein may be stored in the storage 530. In another configuration, the memory 420 is configured to store such software. Software shall be construed broadly to mean any type of instructions, whether referred to as software, firmware, middleware, microcode, hardware description language, or otherwise. Instructions may include code (e.g., in source code format, binary code format, executable code format, or any other suitable format of code). The instructions, when executed by the processing circuitry 510, cause the processing circuitry 510 to perform the various processes described herein.

The storage 530 may be magnetic storage, optical storage, and the like, and may be realized, for example, as flash memory or other memory technology, compact disk-read only memory (CD-ROM), Digital Versatile Disks (DVDs), or any other medium which can be used to store the desired information.

The network interface 540 allows the disease predictor 130 to communicate with, for example, the agent 140.

It should be understood that the embodiments described herein are not limited to the specific architecture illustrated in FIG. 5, and other architectures may be equally used without departing from the scope of the disclosed embodiments.

The various embodiments disclosed herein can be implemented as hardware, firmware, software, or any combination thereof. Moreover, the software is preferably implemented as an application program tangibly embodied on a program storage unit or computer readable medium consisting of parts, or of certain devices and/or a combination of devices. The application program may be uploaded to, and executed by, a machine comprising any suitable architecture. Preferably, the machine is implemented on a computer platform having hardware such as one or more central processing units (“CPUs”), a memory, and input/output interfaces. The computer platform may also include an operating system and microinstruction code. The various processes and functions described herein may be either part of the microinstruction code or part of the application program, or any combination thereof, which may be executed by a CPU, whether or not such a computer or processor is explicitly shown. In addition, various other peripheral units may be connected to the computer platform such as an additional data storage unit and a printing unit. Furthermore, a non-transitory computer readable medium is any computer readable medium except for a transitory propagating signal.

All examples and conditional language recited herein are intended for pedagogical purposes to aid the reader in understanding the principles of the disclosed embodiment and the concepts contributed by the inventor to furthering the art, and are to be construed as being without limitation to such specifically recited examples and conditions. Moreover, all statements herein reciting principles, aspects, and embodiments of the disclosed embodiments, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

It should be understood that any reference to an element herein using a designation such as “first,” “second,” and so forth does not generally limit the quantity or order of those elements. Rather, these designations are generally used herein as a convenient method of distinguishing between two or more elements or instances of an element. Thus, a reference to first and second elements does not mean that only two elements may be employed there or that the first element must precede the second element in some manner. Also, unless stated otherwise, a set of elements comprises one or more elements.

As used herein, the phrase “at least one of” followed by a listing of items means that any of the listed items can be utilized individually, or any combination of two or more of the listed items can be utilized. For example, if a system is described as including “at least one of A, B, and C,” the system can include A alone; B alone; C alone; 2A; 2B; 2C; 3A; A and B in combination; B and C in combination; A and C in combination; A, B, and C in combination; 2A and C in combination; A, 3B, and 2C in combination; and the like.

What is claimed is:

1. A method for predictive disease identification via simulations improved using machine learning, comprising:
applying at least one machine learning model to features extracted from data including animal characteristics data of an animal, wherein outputs of the at least one machine learning model include a plurality of disease predictor values, wherein each disease predictor value corresponds to a respective disease type of a plurality of disease types;
running a plurality of disease contraction simulations based on the plurality of disease predictor values;
generating disease contraction statistics based on results of the plurality of disease contraction simulations; and
determining, based on the disease contraction statistics, at least one disease prediction for the animal.

2. The method of claim 1, wherein the plurality of disease contraction simulations includes a plurality of temporal variation simulations for each of a plurality of respective time periods, wherein the at least one disease prediction indicates a likelihood of contracting each of at least one predicted disease by the animal in each of the plurality of time periods.

3. The method of claim 1, wherein the disease contraction simulations are Monte Carlo simulations.

4. The method of claim 3, further comprising:
creating, for each of the plurality of disease types, a model of possible results based on a probability distribution for the disease type, wherein the probability distribution for each disease type is determined based on the disease predictor value of the plurality of disease predictor values corresponding to the disease type, wherein the Monte Carlo simulations are run using the model of possible results for each disease type.

5. The method of claim 1, wherein the at least one machine learning model includes a plurality of first machine learning models and a second machine learning model, wherein the second machine learning model is a combiner model, wherein the plurality of disease predictor values is a plurality of second disease predictor values, wherein applying the at least one machine learning model further comprises:
applying the plurality of first machine learning models to the features extracted from the data including the animal characteristics data of the animal, wherein outputs of the plurality of first machine learning models includes a plurality of first disease predictor values, wherein each first disease predictor value corresponds to a respective disease type of the plurality of disease types; and
applying a combiner model to the plurality of first disease predictor values in order to output the plurality of second disease predictor values, wherein each second disease predictor value corresponds to one of the plurality of disease types, wherein the combiner model is a second machine learning model trained using a training data set including training outputs for the plurality of first machine learning models.

6. The method of claim 5, wherein the plurality of first machine learning models includes a boosting ensemble of sequentially applied boosting machine learning models and at least one non-boosting machine learning model.

7. The method of claim 5, wherein the plurality of first machine learning models includes a logistic regression model and at least one non-logistic regression model.

8. The method of claim 5, wherein the wherein the plurality of first machine learning models includes a boosting ensemble and a logistic regression model.

9. The method of claim 5, wherein the plurality of disease types includes at least one predetermined group of diseases.

10. A non-transitory computer readable medium having stored thereon instructions for causing a processing circuitry to execute a process, the process comprising:
applying at least one machine learning model to features extracted from data including animal characteristics data of an animal, wherein outputs of the at least one machine learning model include a plurality of disease predictor values, wherein each disease predictor value corresponds to a respective disease type of a plurality of disease types;
running a plurality of disease contraction simulations based on the plurality of disease predictor values;
generating disease contraction statistics based on results of the plurality of disease contraction simulations; and
determining, based on the disease contraction statistics, at least one disease prediction for the animal.

11. A system for predictive disease identification via simulations improved using machine learning, comprising:
a processing circuitry; and
a memory, the memory containing instructions that, when executed by the processing circuitry, configure the system to:
apply at least one machine learning model to features extracted from data including animal characteristics data of an animal, wherein outputs of the at least one machine learning model include a plurality of disease predictor values, wherein each disease predictor value corresponds to a respective disease type of a plurality of disease types;
run a plurality of disease contraction simulations based on the plurality of disease predictor values;
generate disease contraction statistics based on results of the plurality of disease contraction simulations; and
determine, based on the disease contraction statistics, at least one disease prediction for the animal.

12. The system of claim 11, wherein the plurality of disease contraction simulations includes a plurality of temporal variation simulations for each of a plurality of respective time periods, wherein the at least one disease prediction indicates a likelihood of contracting each of at least one predicted disease by the animal in each of the plurality of time periods.

13. The system of claim 11, wherein the disease contraction simulations are Monte Carlo simulations.

14. The system of claim 13, wherein the system is further configured to:
create, for each of the plurality of disease types, a model of possible results based on a probability distribution for the disease type, wherein the probability distribution for each disease type is determined based on the disease predictor value of the plurality of disease predictor values corresponding to the disease type, wherein the Monte Carlo simulations are run using the model of possible results for each disease type.

15. The system of claim 11, wherein the at least one machine learning model includes a plurality of first machine learning models and a second machine learning model, wherein the second machine learning model is a combiner model, wherein the plurality of disease predictor values is a plurality of second disease predictor values, wherein the system is further configured to:
apply the plurality of first machine learning models to the features extracted from the data including the animal characteristics data of the animal, wherein outputs of the plurality of first machine learning models includes a plurality of first disease predictor values, wherein each first disease predictor value corresponds to a respective disease type of the plurality of disease types; and
apply a combiner model to the plurality of first disease predictor values in order to output the plurality of second disease predictor values, wherein each second disease predictor value corresponds to one of the plurality of disease types, wherein the combiner model is a second machine learning model trained using a training data set including training outputs for the plurality of first machine learning models.

16. The system of claim 15, wherein the plurality of first machine learning models includes a boosting ensemble of sequentially applied boosting machine learning models and at least one non-boosting machine learning model.

17. The system of claim 15, wherein the plurality of first machine learning models includes a logistic regression model and at least one non-logistic regression model.

18. The system of claim 15, wherein the wherein the plurality of first machine learning models includes a boosting ensemble and a logistic regression model.

19. The system of claim 15, wherein the plurality of disease types includes at least one predetermined group of diseases.
